(19) United States
(12) Patent Application Publication
(10) Pub. No.: US 2026/0137934 A1
(43) Pub. Date: May 21, 2026

(54) SYSTEM AND METHOD FOR DEEP BRAIN STIMULATION USING FLOATING LINE TRANSCRANIAL ELECTRICAL STIMULATION

(71) Applicant: CARNEGIE MELLON UNIVERSITY, Pittsburgh, PA (US)

(72) Inventors: Maysam Chamanzar, Pittsburgh, PA (US); Vishal Jain, Pittsburgh, PA (US); Mats Forssell, Pittsburgh, PA (US); Pulkit Grover, Pittsburgh, PA (US)

(73) Assignee: CARNEGIE MELLON UNIVERSITY, Pittsburgh, PA (US)

(21) Appl. No.: 19/391,105

(22) Filed: Nov. 17, 2025

Related U.S. Application Data
(60) Provisional application No. 63/721,118, filed on Nov. 15, 2024.

(51) Int. Cl. A61N 1/36; A61N 1/04; A61N 1/05

(52) U.S. Cl. CPC A61N 1/36017; A61N 1/0476; A61N 1/0534; A61N 1/36025

(57) ABSTRACT

Disclosed herein is a system and method for performing floating transcranial electrical stimulation in which one or more free-floating insulated wires or bundles of wires are surgically implanted in the brain. The wires have exposed electrodes at both ends and are coupled with an electrode array applied to an external surface of the skull. The single wire or a bundle of wires acts to relay an electric field generated by the electrode array to a target area deep in the brain, thereby realizing the benefits of conventional deep brain stimulation without the inherent risk of infection associated therewith.

### RELATED APPLICATIONS

This application claims the benefit of U.S. Provisional Patent Application No. 63/721,118, filed Nov. 15, 2024, the contents of which are incorporated herein in their entirety.

### BACKGROUND

Deep brain stimulation (DBS) has long been established as an effective treatment for various neurological disorders, including Parkinson's disease, essential tremor, and dystonia. Traditional DBS systems include electrodes that are surgically inserted into specific regions of the brain, an electrical pulse generator and a wire that connects the pulse generator to the electrodes. The pulse generator is usually implanted in the chest cavity and the wire is routed under the skin. While effective, the surgical procedure is highly invasive, with risks such as infection and hemorrhage. Post-surgical complications, such as such as infection, electrode/lead migration, pulse generator malfunction and skin erosion, can also happen. Many of these complications happen because of the wired connection between the deep brain electrodes and the implanted pulse generator.

Recent advancements in neuromodulation have seen the emergence of non-invasive brain stimulation techniques targeting deeper regions in brain, providing a safer alternative to traditional surgical methods. Among these techniques, focused ultrasound stimulation (FUS) has shown considerable promise. FUS leverages high-intensity sound waves to target specific brain regions without the need for incisions, offering precise modulation of neural activity, but its effectiveness in clinical DBS treatments is not well established. Other traditional non-invasive brain stimulation techniques such as transcranial magnetic stimulation (TMS) or transcranial electrical stimulation (TES) offer promising alternatives to surgical methods but come with their own set of challenges and limitations. Most importantly, the depth and intensity of stimulation achievable by TMS and TES are limited compared to implanted electrodes, essentially restricting their use to surface or cortical brain regions rather than deeper structures involved in the neurological disorders able to be treated by DBS. This is because the electric or magnetic fields enter the brain from the surface and have amplitudes that decay as they travel deeper in the brain. To achieve useful neuromodulatory amplitudes in the depth, the field amplitudes in the shallower brain regions will necessarily be suprathreshold, resulting in side effects and likely severe safety concerns. To increase the depth that can be reached by TES, temporal interference (TI) utilizes two pairs of scalp electrodes to inject sinusoidal currents at slightly offset frequencies to produce neuromodulatory effects in deeper regions without superficial stimulation, but the effectiveness of the technique still unconfirmed.

Another recent avenue to reduce the surgical burden of DBS is the development of miniature stimulators that can be mounted in the skull and therefore do not require placing an implanted stimulator in the chest and connecting it to the DBS leads. However, the size of these devices requires a large craniotomy which still presents high surgical risks.

An alternative approach that has been employed in peripheral nerve stimulation consists of only implanting a passive lead consisting of a stimulation electrode positioned in close proximity to the target nerve at one end and a capture electrode shallowly placed under the scalp at the other end. Current pulses are injected using a wearable stimulator and couple transcutaneously to the implanted lead, resulting in stimulation of the nerve. This technique has been used in clinical trials in multiple conditions targeting peripheral nerves with remarkable safety and efficacy results [ref], but to date no fully passive implant has been demonstrated for DBS. Deep brain stimulation using transcranial methods necessitates a new approach to achieve the required amplitude in deep brain regions while enhancing targeting precision, reducing individual variability, and improving overall safety.

### SUMMARY

Disclosed herein is a novel method, referred to as floating transcranial electrical stimulation (FLOATES). This method is a minimally invasive technique that combines the benefits of DBS and TES to enable deep brain stimulation via transcranial electrodes. FLOATES consists of the surgical implantation of one or more free-floating insulated wires or bundles of wires in the brain, targeting the desired region or regions. The wires have exposed electrodes at both ends. The distal (output) electrode acts similarly to a conventional DBS electrode in locally stimulating a targeted area, and the proximal (input) electrode is located at the top of the brain, near the skull.

The wires may be implanted through a hole in the skull which, is preferably resealed to prevent infection. Following implantation, an electrode array disposed on the scalp is used to non-invasively inject currents into the brain. Focal electric stimulation can be delivered to the brain through the skull non-invasively using high density patterns of electric stimulation on the surface of the scalp. This method enables targeted steerable neural stimulation using non-invasive TES. The use of high-density scalp electrodes for stimulation allows designing focal currents that couple into the wire via the input wire electrode, allowing stimulation through the output electrode. A schematic view of the simplest embodiment of the invention using a single wire and a single electrode array is shown in FIG. 1. The components of the invention are shown in FIG. 2, and consist of microwire bundle 204 (or single microwire) and electrode array 202. As shown in FIG. 2, the wires in the microwire bundle may be held in place using a brace composed of polyethylene glycol (PEG) or another biocompatible material. Preferably, the material used to bind the microwires will dissolve after implantation such that the wires are not connected in the brain. This reduces damage to the brain by reducing the strain caused by large rigid structures that are unable to move along with the brain micromovements.

The disclosed method represents a significant leap in neuromodulation by bridging the gap between existing transcranial electrical stimulation (TES) and deep brain stimulation (DBS). While TES cannot reach the deep brain precisely and DBS is invasive with accessibility limitations, the disclosed method combines the non-invasiveness of TES with the precision of DBS, while offering scalable and enhanced control over the stimulation field. Designed to modulate deep neural circuits involved in complex disorders (e.g., depression), the disclosed method introduces several novel features for neuromodulation therapies. It employs ultra-thin fully implanted microwires or microwire bundles that are neither tethered to backend electronics nor anchored to the skull, thereby reducing tissue damage and the risks associated with implanted hardware in traditional DBS. The disclosed method thus benefits from the safety and flexibility of TES and extends its reach to match that of DBS by relaying and delivering focused stimulation to deep brain structures in a minimally invasive manner, offering a scalable and safer solution for neuromodulation. The surgery is simplified due to not having to connect leads from the brain to an implanted pulse generator. The wearable surface array and the backend hardware are all external. Moreover, the risks of infection are reduced because the wires are fully embedded in the brain without crossing the skull and the opening in the skull used to implant the wires is sealed.

The disclosed method, in some embodiments, utilizes bundles of microwires covering a broader area of deep-brain targets (e.g., NAc and VTA), enabling precise neuromodulation of circuits involved in MHDs by relaying the desired electric field from the surface. The wire bundle covers a larger area than a single DBS lead and utilizes a high-resolution scalp electrode layout. Therefore, the relayed spatial stimulation pattern can be tailored to the patient's specific neural anatomy and therapeutic needs and optimized to reduce side effects. By integrating with high-density transcranial electrical stimulation (TES) electrode arrays on the scalp, the method enables precise steering of electric fields to deep brain targets at a resolution of <0.5 mm. This approach addresses an inherent limitation of TES where the field attenuates and diffuses through superficial tissues, significantly reducing efficacy and focality, as evidenced by a recorded weak electric field (i.e., ∥E∥˜0.08 V/m) at the subthalamic nucleus (STN) when a 1 mA current applied via two transcranial electrodes. The relay mechanism and the unique steering capability of the proposed method enables changing the stimulation conditions over time to optimize treatment and reduce side effects. Lastly, the method enables simultaneous stimulation of multiple brain regions, providing a higher degree of control, potentially improving therapeutic outcomes and reducing side-effects.

### BRIEF DESCRIPTION OF THE DRAWINGS

FIG. 1 is a schematic diagram of a first embodiment of the invention using a single wire and a single electrode array.

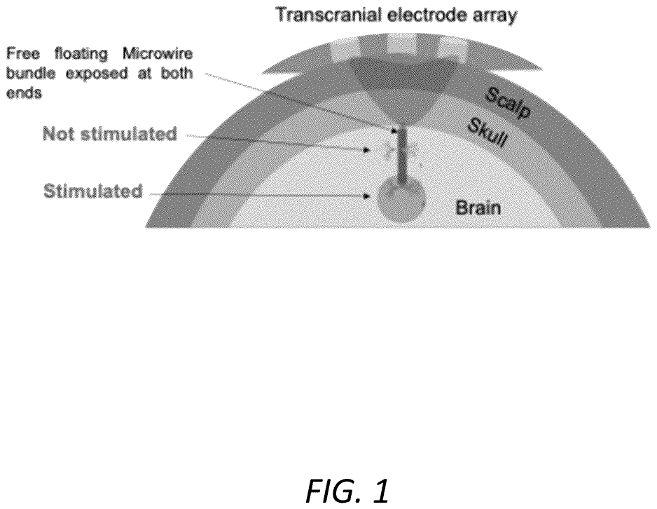

FIG. 2 is a schematic diagram showing the components of the system and the electric field produced at the target.

FIG. 3 illustrates a simulation and benchtop demonstration of current coupling to deep regions of the brain.

FIG. 4 shows the magnitude (V/m) of an electric field measured in saline, normalized to a 1 mA injected current, in three conditions: (a): at the location corresponding to the input of the wire (measured in the absence of the wire), 200 μm from the electrode array; (b): at the location of the wire output in the absence of the wire (scale bar: 100 μm); and (c): at the output of the wire (4.2 mm above the current-injection electrodes).

FIG. 5 illustrates an experimental setup in which the stimulation patch was placed on skull of a rodent and a wire was inserted to target STN and wherein motor response (MEPs) was recorded in contralateral forelimb of the rodent.

FIG. 6 is a series of graphs showing simulations of various parameters on output quantities.

FIG. 7 is an illustration of a second embodiment of the invention using multiple wire bundles.

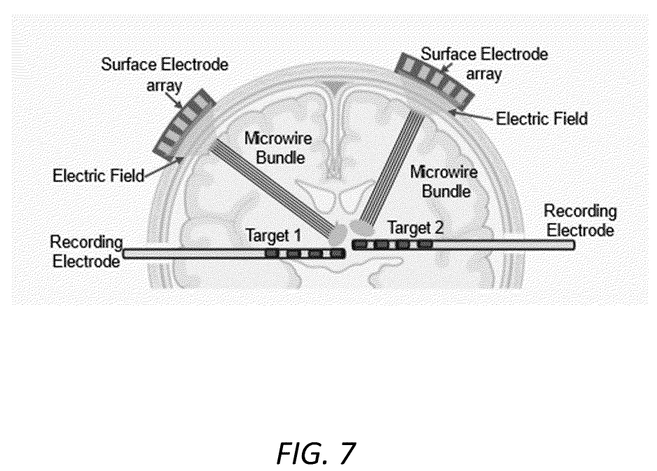

### DETAILED DESCRIPTION

Disclosed herein is a novel method for floating transcranial electrical stimulation that employs an untethered wire or wire bundle to passively couple transcranially injected currents from the brain surface to deep brain regions, achieving focused stimulation deep within the brain. The method delivers significantly higher electric fields to subcortical regions compared to conventional transcranial stimulation approaches. Further, the method requires a significantly lower motor threshold when compared to transcranial stimulation.

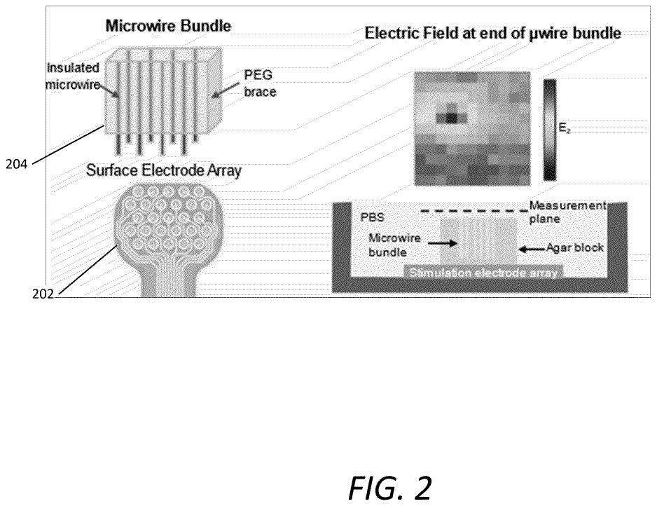

As shown in FIG. 2, the method uses a system comprising two parts: (i) a wearable surface electrode array 202 that generates the transcranial stimulation field and (ii) an untethered, floating single microwire or microwire bundle 204 that relays the stimulation field to the desired targets in the deep brain. The wearable array is manufactured using standard printed circuit board technology and the microwire bundle is fabricated using well-known microfabrication techniques. The resolution of relayed stimulation fields and the steering accuracy is optimized by designing the geometry of the microwire bundle as well as the current injected through the surface electrodes.

The tetherless wire is composed of concentric metal microelectrodes (for example, Model PI2CEA3-200; MicroProbes for Life Science, Gaithersburg, MD, USA). These microwires were used for benchtop and in vivo proof-of-concept experiments. Each wire was inspected under a stereomicroscope to confirm structural integrity and insulation continuity. To achieve different insertion depths suitable for bench and in vivo preparations, the wires were trimmed to lengths ranging from 3.5 mm to 4.0 mm using a wire cutter. Following trimming, the insulation was examined to verify that only the distal tip remained exposed, while the remainder of the shaft maintained intact insulation. Electrodes were cleaned in sterile saline and air-dried prior to use. These length-adjusted electrodes were subsequently used in both benchtop validation studies and in vivo experiments to achieve precise positioning within the target region.

For benchtop testing, the tetherless wire was connected to a motorized manipulator arm and placed in a tub containing 1× phosphate buffered saline (PBS). The bottom of the tub contained a flexible printed circuit board FBCP) consisting of an electrode array 202 as shown in FIG. 2. (e.g., ring electrodes with outer diameter: 0.35 mm and inner diameter: 0.15 mm, arranged in a hexagonal grid at a 0.65 mm pitch). In one embodiment, only a single electrode is used as the anode and the six surrounding electrodes as the cathode. Using the micromanipulator, the floating wire was precisely aligned above the central anode, with the bottom of the wire 200 μm above the electrodes (corresponding to the approximate thickness of a mouse skull). Pulsed current stimulation was applied using biphasic, charge-balance 1 ms/phase pulses with 0.3-1.2 mA injected through the electrodes (the current was adjusted to ensure the recording amplifier was not saturated). The voltage potential created in the PBS was measured using a PBS-filled glass pipette connected to an amplifier and digitizer. The pipette recorded the potential field in a 100 μm grid. The electric field can then be calculated using:

$$\vec{E} = - \vec{∇} V$$

The electric field was recorded at a distance of 4.3 mm from the electrodes (corresponding to 100 μm from the tip of the wire). The electric field was measured both with and without the floating wire. In the absence of the wire, the electric field was also measured 200 μm above the electrodes (corresponding to the location of the input of the wire).

For the proof-of-concept experiments, C57Bl6 mice (12-18 weeks old, both sexes) were used. All mice were randomly distributed among different groups. The mice were maintained on a 12 h light-dark cycle with free access to food and water.

Initial experiments using invasive probes were performed to identify the target region. The target region is defined as the portion of the subthalamic nucleus (STN) that results in motor evoked potentials (MEPs) in the contralateral forelimb when stimulated electrically. C57 mice (n=2) were anesthetized using a ketamine/xylazine cocktail and headfixed on a stereotaxic frame. A microwire electrode (PI2PT30.01A5, Microprobes) was inserted at five locations surrounding the coordinates of the STN (AP=−2.7 mm+−1 mm, ML=1.8 mm+−1 mm). Electrical stimulation (Ripple Grapevine and micro2+stim headstage, Ripple Neuro) was performed using biphasic pulses of 1000 us pulse width injected in trains of 7 pulses at 350 Hz. The injected current amplitude was increased until motor threshold was achieved. Electromyographic recordings were performed using monopolar needles inserted in the contralateral upper limb muscles, connected to a differential amplifier/digitizer recording at 30 kS/s. Motor threshold was characterized using the peak-to-peak amplitude of the MEP following stimulation, as the smallest current able to achieve a MEP amplitude above 50% of the maximum MEP amplitude recorded. At each insertion location, stimulation was delivered at six different depths (DV0.8 mm, 1.5 mm, 2 mm, 3 mm, 4 mm, 5 mm) and the threshold required to evoke MEPs in the contralateral forelimb was measured.

The electrical stimulation consists of waveform generated by a waveform generator coupled to the electrode array such that the waveform causes the electrode array to produce an electric field. In a preferred embodiment, the waveform may consist of pulses of ˜100-1000 mS in width (monophasic or biphasic) which are typically used in deep brain stimulation in clinical applications. Other waveforms (e.g. sinusoidal) could be useful as well.

The method was validated in a mouse model by targeting the STN in the deep brain and recording motor evoked potentials MEPs in upper limbs. Mice (n=4) were anesthetized using a ketamine/xylazine cocktail and head fixed on a stereotaxic frame. An electrode array (ring electrodes with outer diameter: 0.35 mm and inner diameter: 0.15 mm, arranged in a hexagonal grid at a 0.65 mm pitch), with a hole (0.4 mm diameter) at the center was placed on the skull, with the hole above the location of STN (AP=−2.7 mm, ML=1.8 mm from Bregma). The electrode array was connected to a multichannel stimulator. A stimulation pattern utilizing 6 active and 6 return electrodes surrounding the hole was used for stimulation. Stimulation waveform and EMG recording for motor threshold were performed as described in the previous section.

The motor threshold was characterized in three conditions, without moving the electrode array or the recording electrodes: (1) “intact skull” condition: transcranial stimulation through intact skull, without floating wire, as described above; (2) “skull with hole” condition: a hole (0.3 mm diameter) was drilled in the skull through the hole in the electrode array (i.e. at the identified STN location); and (3) “hole+wire” condition: a floating microwire (as above, consisting of the tip of a conical microwire (PI2CEA3-200, Microprobes) cut to a length of 4.5 mm) was inserted through the same hole in the skull, such that its proximal end is flush with the brain surface and its distal end reaches the STN.

Finite element simulations reveal that the efficiency of the method depends on several key parameters including input field strength, wire length and diameter, exposed electrode area, impedance, and tip geometry. The current coupled into the wire scales linearly with the input field, and while larger diameters increase total current flow, the output field amplitude remains relatively constant due to geometric scaling. Increasing the exposed electrode area lowers impedance but decreases local field intensity, suggesting an optimal configuration balancing these effects. Simulations further indicate that reduced electrode impedance and confined input fields maximize coupling efficiency while minimizing off-target activation. The results establish a theoretical and experimental foundation for the method as a minimally invasive, spatially precise brain stimulation platform with potential translational applications in modulating deep neural circuits implicated in neuropsychiatric and movement disorders.

Finite element simulations were performed using the electric currents interface in COMSOL Multiphysics. To mimic the conditions of rodent experiments, a spherical head model was utilized consisting of 3 layers: scalp (radius=10 mm, conductivity=0.465 S/m), skull (radius=9.8 mm, conductivity=0.01 S/m), and brain (radius=9.6 mm, conductivity=0.2 S/m). Circular electrodes (0.4 mm diameter) were placed on the surface of the scalp, matching the arrangement of 7 electrodes used in the benchtop characterization (center electrode placed on the zenith, surrounded by six evenly distributed equidistant electrodes at a 0.65 mm distance). Electric current (1 mA) was injected to the center electrode, with the six surrounding electrodes acting as the ground (and therefore the return electrodes) and the electric field generated in the brain in steady-state was simulated.

A cylindrical floating wire was included (nominal parameters: diameter=0.2 mm, depth from the brain surface=0 mm, length=4 mm, conductivity=106 S/m) with insulation around the sides and exposed at the top (proximal end, or input facet) and bottom (distal end, output facet). Additionally, a portion of the length around the sides near the top and bottom was also exposed (nominal electrode length=0.1 mm for top and bottom). These exposed regions correspond to the metal areas acting as electrodes through which current is coupled into and out of the wire. These electrodes are modeled as a surface impedance (nominal surface resistance: 1 Ω/m). Simulations were performed in the presence and absence of this wire and also while sweeping the design parameters to evaluate the effect of wire properties on the electric field relay capability.

Three metrics were considered to characterize the floating wire performance: (1) the total electric current flowing through the wire; (2) the electric field amplitude at the output of the wire (maximum electric field 100 μm away from the wire); (3) the volume of tissue around the tip of the wire in which the electric field exceeds a chosen threshold of 100 V/m (approximating the stimulation volume).

A simplified analytical model was developed to predict the FLOATES outputs (current coupling and electric field at the output). The mode assumes low coupling between the wire and electric field and calculates the current through the wire based on the potential difference between the input and the output facets of the wire, assuming an existing electric field. Specifically, the model uses as its input an electric field potential in the brain produced in the absence of the floating wire (obtained from a finite element simulation or analytical expressions such as skull transparency). A cylindrical wire is defined by its position within this electric field, dimensions (i.e., radius r and length L), insulation coverage (quantified by the distance of deinsulated metal at the input Lₑₗⁱⁿ and output Lₑₗ^{out} of the wire), bulk conductivity ρ_{wire}, and surface resistance of the exposed metal ρₑₗ. To calculate the current flowing through the wire, Vᵢₙ, the average potential along the exposed region at the input of the wire is calculated, and Vₒᵤₜ, the potential in the middle of the output face of the wire are first calculated. The total wire resistance Zₜₒₜ is the series combination of three resistances: Zᵢₙ, the resistance of the input electrode; Zₒᵤₜ, the resistance of the output electrode; and Z_{wire}, the bulk resistance of wire:

$${Z}_{i n} = \frac{{ρ}_{e l}}{π ( {r}^{2} + 2 r {L}_{e l}^{i n} )}$$

$${Z}_{out} = \frac{{ρ}_{e l}}{π ( {r}^{2} + 2 r {L}_{e l}^{out} )}$$

$${Z}_{w i r e} = \frac{{ρ}_{w i r {e}^{L}}}{π {r}^{2}}$$

$${Z}_{tot} = {Z}_{i n} + {Z}_{w i r e} + {Z}_{out}$$

Additionally, the resistance path from the output of the wire (spreading resistance from the wire output) is approximated as:

$${R}_{ret} = \frac{{ρ}_{b r a i n}}{2 π r}$$

Then the current flowing through the wire is:

$${I}_{wire} = \frac{{V}_{i n} - {V}_{out}}{{Z}_{tot}} ( 1 + \frac{{R}_{ret}}{{Z}_{tot}} )$$

The electric field at the output of the wire is then calculated by assuming a constant current density along the entire exposed electrode at the output facet:

$${J}_{out} = \frac{{I}_{wire}}{π ( {r}^{2} + 2 r {L}_{e l}^{out} )}$$

The electric field at a point (x,y,z) is calculated based by integrating the distance between this point and the exposed metal electrode area of the output facet.

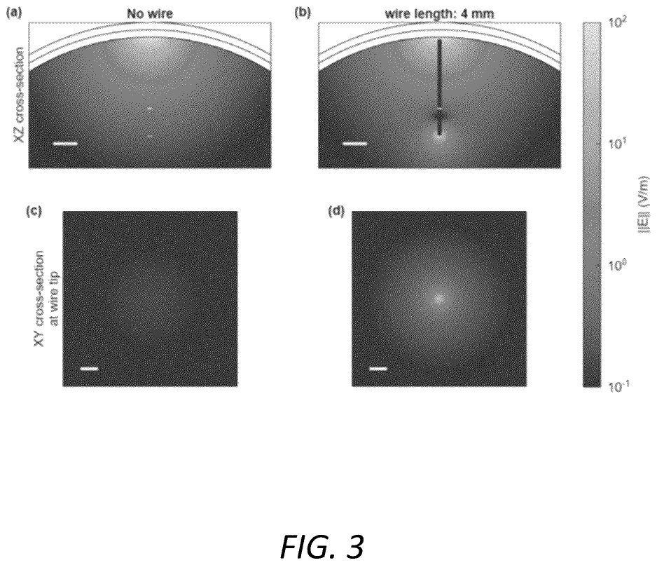

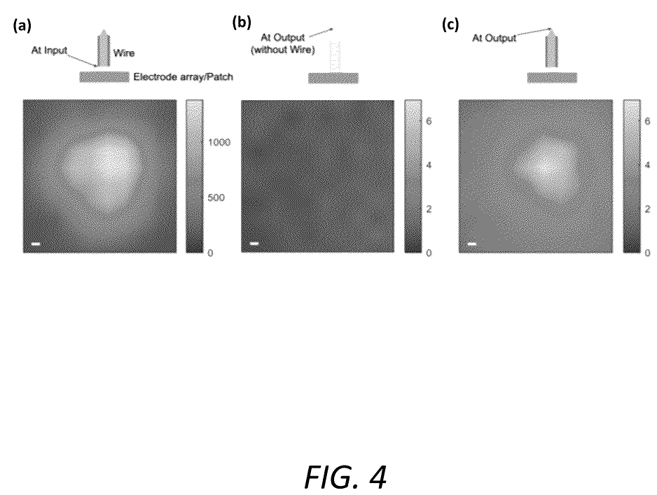

Finite element simulations, illustrated in FIGS. 3(a-d), show that the method can elicit significantly higher electric fields to deep brain regions compared to transcranial electrical stimulation in the absence of the implanted wire. FIGS. 3(a,c) show the electric field produced by a transcranial scalp electrode array and FIGS. 3(b,d) show the electric field produced by relay using a floating wire (diameter=200 μm, length=4 mm, scale bar: 1 mm). The concept was also validated in the benchtop setting using an electrode array placed at bottom of a chamber filled with PBS. A floating wire was suspended in the solution 200 μm above the electrodes to mimic the thickness of a mouse skull. FIGS. 4(a-c) show the electric field measured at the proximal (input) and distal end (output) of the wire. The results indicate 7X enhancement in the electric field at the output when the wire is present (7 V/m), compared to the condition without the wire (1 V/m), measured at the same distance (4.2 mm from the electrodes) (FIGS. 4(b-c)). The measurements illustrate a localized field around the wire output, demonstrating the effectiveness of the wire in maintaining consistent field strength around the output region. As shown in FIG. 4(d), without the wire, however, only a weak diffuse field can be observed due to the distance from the electrodes, highlighting the wire's role in conducting current to deeper regions.

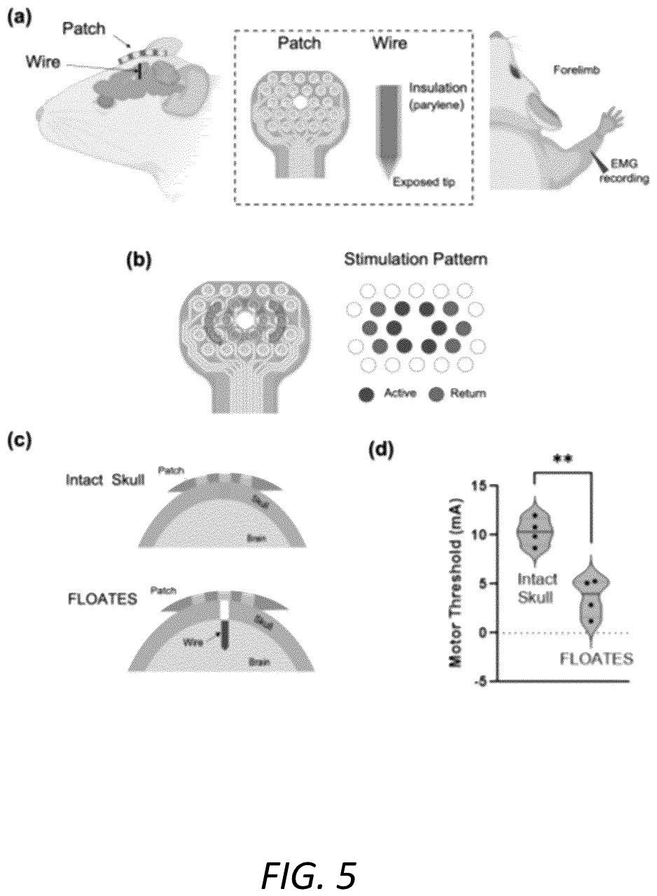

For demonstration of the method in vivo, one challenge is to identify deep stimulation targets to insert the floating wire to elicit motor-evoked potentials (MEP). MEP responses can be elicited by stimulating the subthalamic nucleus (STN) in the deep brain (AP-2.7 mm, ML1.8 mm, DV4.5 mm). Because the transcranial currents that couple into the wire can also affect superficial cortical regions, the wire insertion location must be such that stimulation of the superficial regions at the insertion location does not cause motor activity. This is critical, especially since we have previously demonstrated non-invasive stimulation of motor cortex in mice using surface electrodes to evoke MEPs in forelimbs. To avoid motor cortex stimulation, the chosen region for insertion (AP=−2.7 mm, ML=1.8 mm) corresponds to the visual cortex V2. A baseline intracortical micro-stimulation (ICMS) experiment was conducted in which a microwire stimulation probe was inserted at 5 different locations surrounding the coordinates of STN (i.e., AP-2.7 mm+−1 mm, ML 1.8 mm+−1 mm, as shown in FIG. 5(a). At each insertion location, stimulation was delivered at six different depths (DV0.8 mm, 1.5 mm, 2 mm, 3 mm, 4 mm, 5 mm) and the threshold required to evoke MEPs in the contralateral forelimb was measured. FIG. 5(b) shows the current injection pattern showing a circular arrangement of anode (red) electrodes surrounded by cathode (blue) electrodes.

Results revealed that the extent of the deep brain region in which stimulation can evoke motor responses spreads in AP and ML by 0.5 mm and in DV by 1 mm. The region is confined to depths greater than 4 mm, as no motor response was observed by stimulating more superficial layers (i.e., cortical and thalamic regions) FIG. 5(c) shows the two experimental conditions, showing an intact skull and the skull using the present method. This location was used for all subsequent experiments to demonstrate the method.

The method was validated in a mouse model by targeting STN in the deep brain and recording MEPs. Transcranial currents were injected into the brain using an electrode array placed on the mouse skull and MEPs were recorded using EMG needle electrode inserted in the contralateral forelimb, as shown in FIG. 5(a). A floating wire was inserted into the deep brain targeting STN to demonstrate neuronal stimulation deep within the brain (FIG. 5(b)). In each animal, two different conditions were tested: 1) transcranial stimulation without floating wire and an intact skull, and 2) transcranial stimulation with FLOATES wire inserted through the same hole in the skull.

As shown in FIG. 5(d), results (n=4) revealed that FLOATES reduced the motor threshold by 3× (3.6 mA±0.97 mA; Mean±SEM) when compared to stimulation through intact skull (10.33 mA±0.70 mA) with significance of p=0.0019.

Benchtop testing and in vivo animal experiments establish the feasibility of relaying a focused electric field current through floating wires. As illustrated in FIG. 3(b), the method can deliver significantly higher electric field to deep brain regions compared to traditional noninvasive electrical stimulation. The rich design space of the FLOATES system enables optimizing the electric current coupling efficiency and the relaying mechanism. To understand the effects of different parameters on the performance of this system and inform future design and optimization strategies, we have performed rigorous

Finite Element Method (FEM) simulations were conducted to study the effect of the input field, the wire dimensions (length and diameter), the area of exposed electrodes, the electrode impedance and the tip geometry. We considered three output metrics of (i) electric current amplitude flowing through the wire, (ii) the electric field amplitude at the output of the wire, and (iii) the volume of tissue in which the local electric field exceeds a chosen threshold (approximating the stimulation volume). In addition to the FEM simulation, an analytical model was developed which decouples the wire from the electric field (i.e. assumes that the presence of the wire does not affect the electric field generated by the stimulation electrodes). In addition to providing insight into the interactions between the wire and the electric field, this analytical model, due to its simplicity, can be used for optimization studies.

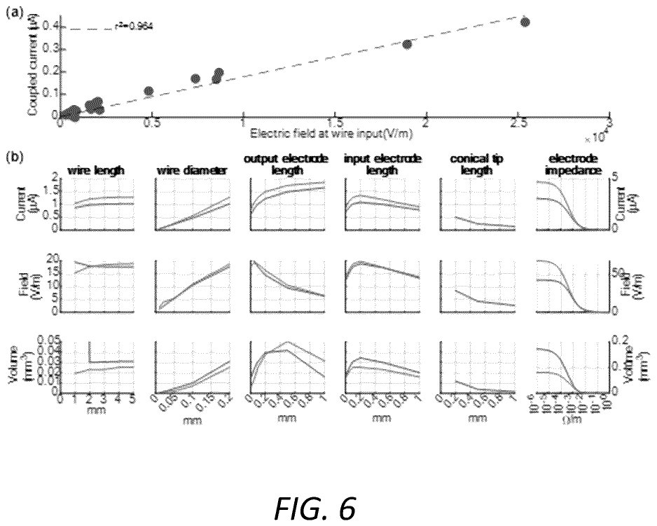

FIG. 6(a) shows, for a nominal wire geometry, the current coupled through the wire as a function of the local field at the distal end of the wire (i.e. at the input electrode) calculated in the absence of the wire. The plot shows a good linear fit, meaning that, for a given wire, the current that flows through the wire is proportional to the field at the location corresponding to the input electrode. Therefore, the optimization of the wire parameters and the currents injected through the scalp are decoupled. Regardless of the wire properties, the current coupled into the wire will be maximized if we maximize the electric field at the input facet. This observation makes sense if the wire is considered as a perturbation to the applied transcranial electric field, that is, if the current coupled into the wire is small. If a large current couples to the wire, then the wire perturbs the applied electric field more strongly, and the effects of the wire on the electric field cannot be decoupled. From the perspective of deep brain neural stimulation, in addition to coupling to the wire, the applied electric field results in off-target superficial stimulation. Therefore, the optimization of the currents injected through the scalp (which elicit the electric field in the brain) should take into account both the amplitude of the electric field at the wire input (to maximize the current flowing into the wire) and the electric field at other superficial locations. In practice, a confined input field is desirable to reduce off-target effects, but this comes at the cost of reduced maximum amplitude. Effects of the wire geometry on the output quantities are shown in the graphs in FIG. 6(b).

Both the current coupled to the wire and the output field indicate that unless the wire is very short (<=1 mm), the output is not affected by the length of the wire. This is expected due to the high conductivity of the metal wire (i.e., ^{˜}one million times larger than the surrounding tissue). For short wire lengths, the electric field at the output is larger because the effect of the applied electric field (resulting from the injected current through the scalp, without coupling to the wire) is still noticeable at the output.

Increasing the wire diameter reduces the wire impedance, and thereby increases the current coupled into the wire. While the current flowing into the wire is proportional to the diameter, the output field increases very little. This is because the output field is also inversely proportional to the area of the output electrode (which scales quadratically with the diameter).

The exposed area of the electrodes at both the input and output ends can also be varied. For a cylindrical wire, increasing the exposed electrode area is achieved by increasing the length of the de-insulated portion of the wire. For the output electrode, increasing the exposed area reduces the overall wire impedance and therefore increases the coupling of current into the wire. However, the electric field at the output is inversely proportional to the exposed area; therefore, overall increasing the area reduces the electric field at the output. For the input electrode, increasing the exposed area also reduces the impedance, and therefore allows more current to couple into the wire. However, the benefits are limited because with larger electrode area, the average electrode depth also increases (because the area is increased by exposing a longer portion of the wire). Because the applied electric field decays with depth, the result is that a lower average electric field couples into the wire. Therefore, there is an optimal exposed input area to maximize the current coupled into the wire.

Utilizing a conical tip, rather than a perfect cylinder reduces the current coupling and output field because the electrode area is reduced, thereby increasing the impedance of the wire.

The electrode impedance is modeled as a surface resistance on the exposed metal area and is therefore proportional to the surface resistance ρₑₗ. Coupled current, output field and stimulation volume all decrease with increasing impedance. In practice for a given electrode size, the impedance can be controlled in two ways: by changing the electrode material or depositing a surface coating (e.g. PEDOT) to reduce the electrode-tissue impedance, or by changing the injected signal through the electrode. The simulations that were performed used steady-state models, and therefore, did not take into account the temporal properties of the signal. However, real microelectrodes behave like a capacitor, therefore their impedance is inversely proportional to the frequency of the signal. Using shorter stimulation pulses increases the characteristic frequency, and therefore, reduces the effective impedance.

Further the simulation characterization of the method revealed several key insights into its efficiency for deep brain stimulation. First, the study demonstrated that the current coupled into the wire is proportional to the input electric field, confirming that optimizing the wire design and input field strength are separate tasks. This decoupling allows for better control over the stimulation by focusing on enhancing the field at the input electrode. However, care must be taken to avoid excessive off-target stimulation, a challenge commonly faced in non-invasive brain stimulation techniques like transcranial electrical stimulation (TES).

For the wire, the major factor influencing the efficiency is the overall wire impedance, which is dominated by the input and output electrode impedances. The electrode impedances can be reduced by increasing the exposed metal area, which for a cylindrical wire is achieved either through increasing the wire diameter or by increasing the length of the exposed area along the wire. For the input electrode, a wide conductive electrode resting on the brain surface could be used, increasing the coupling efficiency as well as simplifying wire explantation. However, the output electrode impedance will then dominate the overall wire impedance. Reducing the output electrode impedance through geometric means (i.e., increasing the exposed metal area of the output electrode) will increase the current flowing through the wire but result in lower electric field at the output (because the amplitude of the electric field at the output of a wire is inversely proportional to the electrode area). Reducing the impedance can also be achieved through improved electrode-tissue interface (e.g., through PEDOT electrodeposition), without changing the geometry of the electrodes.

The wires used in the single wire embodiment, or the wire bundles embodiment may comprise commercially-available microwires having concentric microelectrodes (i.e., a metal wire at the center, surrounded by an insulating layer surrounded by a second metal sheath). In other embodiments, the wires may be single microwires having a layer of insulation. In one embodiment, the wires in the wire bundle may couple to electrodes in the electrode array on a one-to-one basis. Alternatively, the wire bundle may be denser than the electrode array (i.e., more wires in the wire bundle than there are electrodes in et ne3lectrode array).

Variations of the above-disclosed and other features and functions, or alternatives thereof, may be combined into many other different systems, devices, methods or applications. Variations presently unforeseen or unanticipated alternatives, modifications, variations or improvements therein may be subsequently made by those skilled in the art, each of which is also intended to be encompassed by the disclosed embodiments and the appended claims. Many known variations are contemplated to be within the scope of the invention. In some embodiments, only a single microwire is used, while in other embodiments, a wire bundle is used. In the wire bundle embodiment, wires in the bundle may be different lengths. In a second embodiment of the invention, multiple wires or wire bundles, coupled with multiple electrode arrays, may be used to target multiple areas or may be used in a beamforming configuration to steer the electric field to a single target area through interaction of the electric fields produced by each wire bundle.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent devices and apparatuses within the scope of the disclosure, in addition to those enumerated herein, will be apparent to those skilled in the art from the descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular materials, compounds, compositions or alloys, which can, of course, vary. It is also to be understood that the terminology used herein is for the purpose of describing particular embodiments only and is not intended to be limiting.

We claim:

1. A system for deep brain stimulation comprising:
a wire or wire bundle implanted in a brain of a subject, a distal end of the wire or wire bundle disposed at a target area deep in the brain and a proximal end of the wire or wire bundle disposed at or near the surface of the brain; and
an electrode array disposed on a surface of the scalp or the skull of the subject and coupled to the wire or wire bundle such that an electric field generated by the electrode array is relayed from the proximal end of the wire of wire bundle to the distal end of the wire or wire bundle.

2. The system of claim 1 further comprising a wave generator coupled to the electrode array.

3. The system of claim 1 wherein the electrode array comprises a plurality of electrodes, one or more electrodes being anodes and one or more electrodes being cathodes.

4. The system of claim 1 wherein the wire or each wire in the wire bundle have an exposed tip at the proximal end, an exposed tip at the distal end and insulation between the tips.

5. The system of claim 1 wherein the wire bundle comprises a plurality of wires held in a bundle by a biocompatible material.

6. The system of claim 2 wherein each wire in the wire bundle may be of different lengths.

7. The system of claim 1 wherein multiple wires or wire bundles are implanted into the brain of the subject.

8. The system of claim 7 further comprising:
multiple electrode arrays coupled to respective wires or wire bundles.

9. The system of claim 1 wherein the electrode array comprises a plurality of electrodes defined on a flexible printed circuit board.

10. The system of claim 7 wherein multiple electric fields produced by the multiple wires or wire bundles interact with each other.

11. The system of claim 7 wherein multiple electric fields produced by the multiple wires or wire bundles target independent areas of the brain.

12. A method comprising:
implanting a wire or wire bundle in a brain of a subject through a hole defined in the skull of the subject;
placing an electrode array in proximity to the hole such that an electric field generated by the electrode array couples transcutaneously to the wire or wire bundle.

13. The method of claim 12 further comprising:
sealing the hole defined in the skull after implantation of the wire or wire bundle using a biocompatible material having conductivity similar to the skull of the subject.

14. The method of claim 12 further comprising:
generating a waveform using a waveform generator coupled to the electrode array such that the electrode array produces the electric field.

15. The method of claim 12 wherein a distal end of the wire or wire bundle is disposed at a target area deep in the brain and a proximal end of the wire or wire bundle is disposed at or near the surface of the brain.

16. The method of claim 12 wherein the electrode array comprises a plurality of electrodes, one or more electrodes being anodes and one or more electrodes being cathodes.

17. The method of claim 12 wherein the wire or each wire in the wire bundle have an exposed tip at the proximal end, an exposed tip at the distal end and insulation between the tips.

18. The method of claim 12 wherein the wire bundle comprises a plurality of wires held in a bundle by a biocompatible material.

19. The method of claim 12 further comprising:
implanting multiple wires or wire bundles into the brain of the subject through multiple holes.

20. The method of claim 19 further comprising:
coupling multiple electrode arrays to respective wires or wire bundles.

21. The method of claim 19 wherein multiple electric fields produced by the multiple wires or wire bundles interact with each other.

22. The method of claim 19 wherein multiple electric fields produced by the multiple wires or wire bundles target independent areas of the brain.
